(19) United States
(12) Patent Application Publication
(10) Pub. No.: US 2005/0203509 A1
(43) Pub. Date: Sep. 15, 2005

(54) Device and method for fixing bone segments

(76) Inventors: Anboo Chinnaian, Kuala Lumpur (MY); Jay Pathy, Medina, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/798,834

(22) Filed: Mar. 10, 2004

(51) Int. Cl. A61B 17/56

(52) U.S. Cl. 606/54

(57) ABSTRACT

Embodiments of the fixation device are employed to affix two or more segments of bone in a desired spatial relationship. Embodiments of the fixation device secure and maintain a bone fracture or fractures in proper alignment during the healing process, as well as permit slight movement or micro-motion therebetween to promote healing. In some applications, embodiments of the fixation device place the bone segments under continuous and adjustable compression or distraction while still allowing slight movement or micro-motion at the bone segment interface.

### FIELD OF THE INVENTION

This invention relates generally to surgical devices for fixation of two or more segments of tissue, and more particularly, to a fixation device useful for affixing two or more segments of bone in a desired spatial relation.

### BACKGROUND OF THE INVENTION

The use of both internal and external fixation devices to prevent major movement between two or more sections of bone is known in the art. External casts and braces have commonly been employed to prevent movement between larger bone segments, and have been particularly useful in the fixation of long bone fractures in the extremities. For small bone segments and bones within the trunk of the body which may not be readily immobilized by braces and casts; plates, screws, nails, and wires have been implanted to maintain the relative position of these bones during the healing process.

In some instances, a single screw with a uniform shaft is screwed between two bone segments to maintain the bone segments in place. Alternatively, a pinning device such as a nail may be driven into both of the bone segments to create fixation. In other applications, a lag screw is utilized with a first threaded portion passed through a first bone segment and threaded into a second segment of bone. A nut is then threaded onto a second machine threaded portion of the lag screw to reduce the fracture between the two bone fragments. Alternatively, a similar type of lag screw may be utilized in conjunction with a plate. Each of a series of lag screws is inserted into bone fragments and a plate is attached across the machine-threaded portion of the lag screw. A nut is threaded onto each screw thereby attaching the plate to the bone and maintaining the spatial relationship of the bone fragments. For smaller bones and bone fragments, small wires, commonly known as Kirschner wires (K-wires), have been inserted into the bone to immobilize the fragments. After the wires are inserted, the proximal section of the wire is cut to the desired length.

While these devices have been generally successful, at least initially, in accomplishing the desired immobilization, there are a number of problems that have been encountered with their use. For instance, it has been found that, when used with known fixation devices, K-wires can create an undesirably tight bond between bone fragments.

### SUMMARY OF THE INVENTION

Embodiments of a fixation device are employed to affix two or more segments of bone in a desired spatial relationship. Embodiments of the fixation device secure and maintain a bone fracture or fractures in proper alignment during the healing process, as well as permit slight movement or micro-motion therebetween to promote healing. In some applications, embodiments of the fixation device place the bone segments under continuous and adjustable compression or distraction while still allowing slight movement or micro-motion at the bone segment interface.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1 is a side view of one embodiment of a fixation device formed in accordance with the present invention, wherein the fixation device provides support between first and second bone segments on opposite sides of a bone fracture;

FIG. 2 is a perspective view of the fixation device depicted in FIG. 1 showing the fixation assembly in an assembled state;

FIG. 3 is a perspective view of the fixation device depicted in FIG. 1 showing the fixation assembly in an exploded state;

FIG. 4 is a side view of the fixation device of FIG. 2 depicting the results of an external force F being applied to the support shafts;

FIG. 5A is an end view of the fixation device of FIG. 2 depicting the results of an external force F being applied to the support shaft;

FIG. 5B is another end view of the fixation device of FIG. 2 depicting the results of an external force F being applied to the support shaft in the opposite direction of FIG. 5A;

FIG. 6 is a perspective view of an alternate embodiment of the fixation device formed in accordance with the present invention depicting the fixation device in an assembled state;

FIG. 7 is a perspective view of the fixation device of FIG. 4 showing the fixation device in an exploded state;

FIG. 8 is a perspective view of another alternative embodiment of the fixation device formed in accordance with the present invention depicting the fixation device in an assembled state;

FIG. 9 is a perspective view of the fixation device of FIG. 6 showing the fixation device in an exploded state;

FIG. 10 is a side view of another alternative embodiment of the fixation device formed in accordance with the present invention depicting the fixation device in an assembled state; and

FIG. 11 is a side view of another alternative embodiment of a fixation device formed in accordance with the present invention, wherein the fixation device provides support between first and second bone segments on opposite sides of a bone fracture.

### DETAILED DESCRIPTION OF THE PREFERRED EMBODIMENT

The present invention is generally directed to fixation devices for affixing two or more segments of bone in a desired spatial relationship. More specifically, the present invention is directed to fixation devices that secure and maintain a bone fracture or fractures in proper alignment during the healing process, as well as permitting slight movement or micro-motion therebetween to promote healing. The present invention may include embodiments that utilize a fixation device that places the bone segments under continuous and adjustable compression or distraction while still allowing slight movement or micro-motion at the bone segment interface.

For the purposes of promoting an understanding of the principles of the invention, reference will now be made to the embodiments illustrated in the drawings, and specific language will be used to describe the same. It will nevertheless be understood that no limitation of the scope of the invention is thereby intended, such alterations and further modifications in the illustrated devices, and such further applications of the principles of the invention as illustrated therein being contemplated as would normally occur to one skilled in the art to which the invention relates.

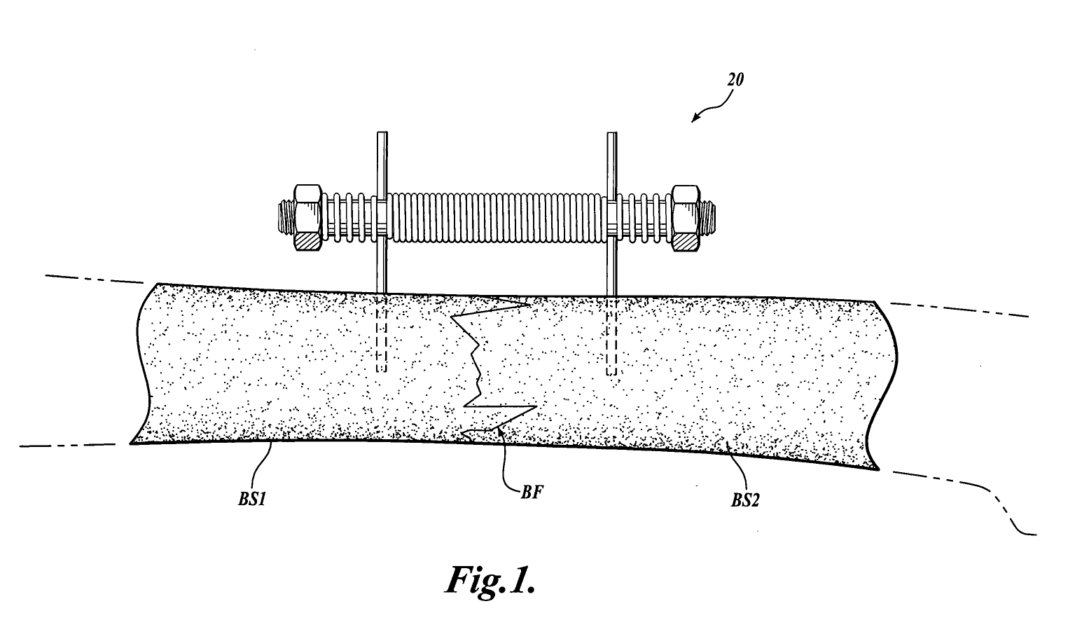

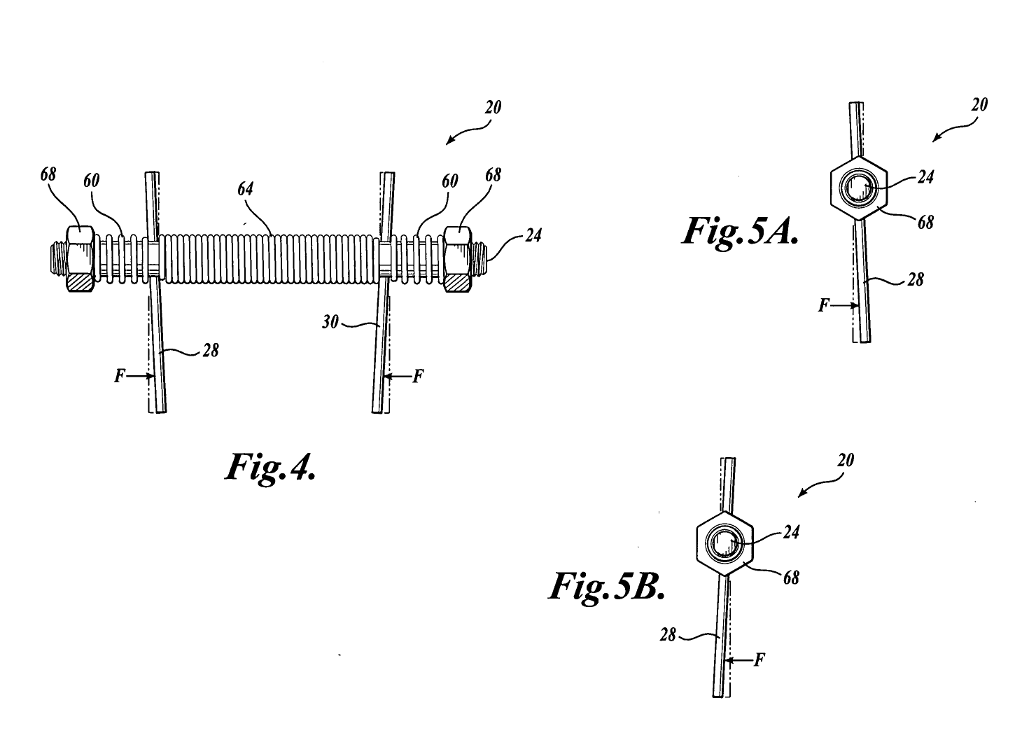

FIGS. 1-5B illustrate one embodiment of a fixation device 20 formed in accordance with the present invention. Referring to FIG. 1, a bone fracture BF is disposed between first and second bone segments BS1 and BS2. The fixation device 20 affixes the first and second bone segments BS1 and BS2 together for potentially providing compression or distraction, preventing major displacement of the bone segments and to support the fractured bone segments during healing. As will be described in more detail below, the fixation device 20 is configured to controllably permit micro-motion between the bone segments BS1 and BS2, thereby promoting the healing process and reducing healing time.

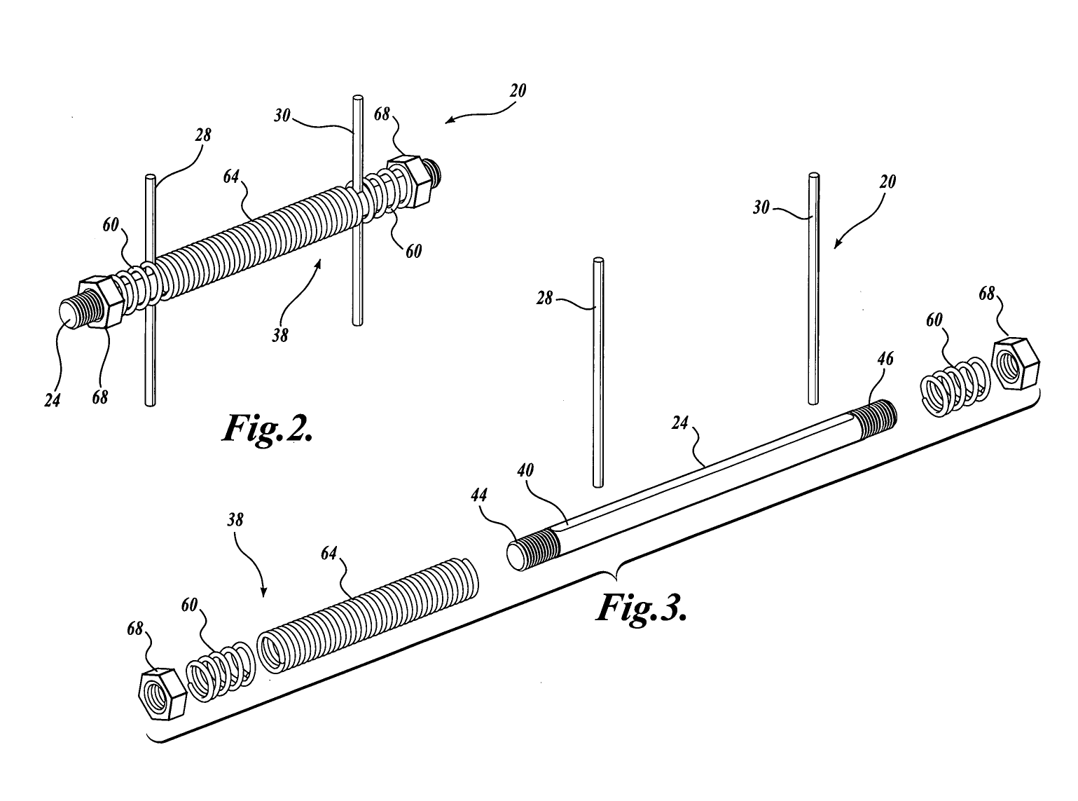

The components of one embodiment of the fixation device 20 will now be described in detail with reference to FIGS. 2 and 3. The device 20 includes a fixating body or support member 24, at least two support pins or shafts 28 and 30, and a dynamic securement assembly 38 for dynamically securing the support shafts 28 and 30 to the support member 24 in a desired spatial relation. The support member 24 is preferably a straight elongate structure substantially cylindrical in shape. The support member 24 includes a slot 40 disposed along a substantial portion of the length of the support member 24. the slot 40 passes through the support member 24. The ends of the support member 24 may be threaded to form opposing threaded ends 44 and 46 for cooperatively engaging securement fasteners, as will be described in detail below.

Although the support shaft 24 is illustrated and described as being both straight and cylindrical, it should be apparent to those skilled in the art that other shapes are suitable for use with and are contemplated to be within the spirit and scope of the present invention. For instance, the support shaft 24 may be arcuate or curvilinear in shape, or rather than being cylindrical in shape, having a cross-section of any selected geometric shape, such as polygonal, oval, etc, without departing from the spirit and scope of the present invention. In the latter configurations, it will be appreciated that clamps or other like devices may be used as securement fasteners, which will be described in more detail below.

The fixation device 20 further includes at least two support shafts 28 and 30 dynamically secured to the support member 24. The support shafts 28 and 30 at their ends are adapted to couple to bone segments. In one embodiment, the distal ends of the support shafts 28 and 30 may be both pointed and threaded to facilitate the coupling of their distal ends to the bone segments. The support shafts 28 and 30 may be made of a material that is amenable to sterilization and is hypoallergenic, a few suitable examples being implant steel and titanium alloy. In one embodiment, the support shafts 28 and 30 may be wires of the smooth or threaded Kirschner type with a diameter of approximately 1.1-1.4 mm. The ends of the Kirschner type wires (K-wires) may be tapered to a point or have other configurations well known in the art.

When the fixation device 20 has been assembled, the support shafts 28 and 30 have been routed through the slot 40 and are disposed in a spaced-apart manner. The slot 40 and the support shafts 28 and 30 are dimensioned and configured to allow each support shaft 28 and 30 to pass therethrough and to freely move along the slot 40 with respect to one another. In the embodiment shown, the support shafts 28 and 30 are positioned such that the support shafts 28 and 30 are substantially perpendicular to the support member 24; however, in other embodiments, the support shafts 28 and 30 may be oriented at any angle with respect to one another, as desired by the treating physician.

The fixation device 20 further includes a dynamic securement assembly 38 for selectively connecting the support shafts 28 and 30 to the support member 24 at controllably adjustable positions along the slot 40 of the support member 24. The dynamic securement assembly 38 comprises a combination of biasing components 60, such as dynamic springs (i.e., their lengths may either be lengthened or shortened by an application of force in the appropriate direction), at least one spacer component 64, such as a static spring (i.e.; its length cannot be substantially shortened, only lengthened by an application of force in the appropriate direction), and securement fasteners 68, such as threaded locking nuts.

As best shown in FIG. 2, the spacer component 64 is disposed between the support shafts 28 and 30 for substantially maintaining a minimum separation distance between the support shafts 28 and 30. The separation distance is application dependent and is typically selected during or just prior to the fixating procedure performed by the physician and/or technician. In the illustrated embodiment, the spacer component 64 is a static spring; however, a single or a plurality of space components or static springs having the desired cumulative length may be used. The spacer component 64 includes an inner passage (not shown) adapted to receive at least a portion of the support member 24 therein.

To provide a dynamic connection between the support shafts 28-30 and the support member 24, biasing components 60 are disposed on the outside of the support shafts 28-30 and are held in place by the securement fasteners 68, which are adapted to be cooperatively engaged or locked at the threaded ends 44 and 46 of the support member 24. As such, the biasing components 60 exert a longitudinally inward directed biasing force against the support shafts 28 and 30 at a selected separation distance with respect to one another and permits the support shafts 28 and 30 to move outwardly in a controlled manner along the slot 40 upon application of an external force against one of the support shafts 28 and 30 in a direction substantially parallel to the longitudinal axis of the support member 24 that is sufficient to overcome the biasing forces exerted against the support shafts. Stated differently, the support shafts 28 and 30 are elastically interconnected by the arrangement of the biasing components. By allowing the support shafts 28 and 30 to move outwardly against the biasing forces of the biasing components 60, the dynamic coupling between the support shafts 28 and 30 and the support member 24 permits micro-motion between the bone segments during use, which in turn, promotes healing of the bone fracture. It will be appreciated that in some embodiments, the spring constants for the biasing components may be either substantially identical or different, depending on application of the fixation device.

In the illustrated embodiment, the biasing components 60 are dynamic springs and the securement fasteners 68 are threaded locking nuts. The dynamic springs each have an inner passage adapted to receive at least a portion of the support member 24 therein. When assembled, the support shafts 28 and 30 are releasably clamped between the spacer 64 and the biasing components 60 by axial movement of the securement fasteners 68 inwardly from the ends of the support member 24.

The securement fasteners 68 may be any device or component that can adjustably hold the biasing components 60 against the support shafts 28 and 30. In the illustrative embodiment, the securement fasteners 68 are threaded locking nuts that cooperatively engage the threads of the support member 24; however, other devices, such as clamps, that can move axially along the support member 24 and adjustably hold the biasing components 60 against the support shafts 28 and 30 may alternatively be used. Additionally, although the illustrated embodiments are depicted and described as using springs as the spacer components and the biasing components, it should be apparent to those skilled in the art that other spacer components and biasing components are suitable for use with and are within the spirit and scope of the present invention. For example, the spacer components 64 may include plastic or metal tubing, washers, etc. and the biasing components 60 may include Belleville washers, resilient compression components made from resilient material, such as rubber, synthetic rubber, etc. or any other presently known or future developed component that exerts a linear or non-linear force when either lengthened or shortened by an applied force. It will also be appreciated that embodiments of the present invention may utilize biasing components that have identical or different spring constants, as may be determined by the physician and/or technician for each application.

The operation of the fixation device 20 will now be described with reference to FIGS. 1-5B. First, the fractured bone is reduced using any known closed or open reduction technique for restoring the overall anatomic alignment of the fractured bone. Next, the support shafts 28 and 30 are inserted into the bone segments BS1 and BS2 disposed on each side of the fracture BF by the use of known medical procedures and secured thereto. In an embodiment where K-wires are utilized as the support shafts 28 and 30, the wires may be bored into the surface of the bone segment. The support shafts 28 and 30 are inserted into the bone segments BS1 and BS2 substantially perpendicular (i.e. approximate right angle) to the longitudinal axis of the bone, although other angles are contemplated to be within the scope of the present invention.

Next, the support member 24 is inserted through the interior passage of one or more spacer components 64. The length of the spacer component is selected to match the separation distance between the first and second support shafts 28 and 30. The length may be obtained by any suitable means, such as by cutting the spacer component 64 to the correct length, or by selecting a spacer component 64 precut to the correct length, etc. It will be appreciated that the selected length is typically less than the total length of the slot 40 to allow outward movement of the shafts 28 and 30.

The support member 24 is then positioned such that the support shafts 28 and 30 are inserted through the slot 40 of the support member 24, wherein the spacer component 64 is positioned in-between the support shafts 28 and 30. The biasing components 60 are then positioned over the ends of the support member 24 such that one of the biasing component 60 rests against each of support shaft 28 and 30. The securement fasteners 68 are then threaded over the threaded ends 44 and 46 of the support member 24. The securement fasteners 68 are then selectively tightened to create a desired amount of pressure or clamping force on the support shafts 24 and 28 by the biasing components 60 at the spacer component 64 to releasably couple the shafts 28 and 30 the support member 24. The selected amount of biasing force determined by the location of the securement fasteners in conjunction with the selected length of the spacer component may result in compression or distraction of the bone fracture. In embodiments of the present invention, the components are configured and arranged such that compression or distraction occurs at the fracture site. This step of selectively tightening the securement fasteners is preferably conducted under the aid of a well known image intensifier.

In one embodiment, the securement fasteners 68 are tightened to positions on the support member 24 such that the support shafts 28 and 30 bias the bone segments BS1 and BS2 together with a force of approximately 80 lbs. per square inch (psi). Although this example is used to illustrate one embodiment of the present invention, it will be appreciated that the securement fasteners 68 can be selectively positioned to hold the bone segments with any desired compression force, depending on the application of the device and the spring constants of the biasing components.

Once the fixation device is secured in place to treat the bone fracture, slight movement of the support shafts 28 and 30 are permitted against the biasing forces of the biasing components 60, which in turn, permits micro-motion between the bone segments affixed by the fixation device 20 upon application of sufficient external force generated by the user during use of the fixation device.

It will be appreciated that during bone healing/reunification, the bone segments may move inwardly with respect to one another. In the embodiments of the present invention, the dynamic coupling of the support shafts to the support member allow the support shafts to move inward at their distal ends (i.e., the end connected to the bone segments), thus providing stress yielding, as opposed to prior art rigid fixation devices wherein the bone segments have to “pull” the ends of the support shafts together during the healing process. This condition found in the prior art is typically referred to as stress shielding and is the main cause of implant failure.

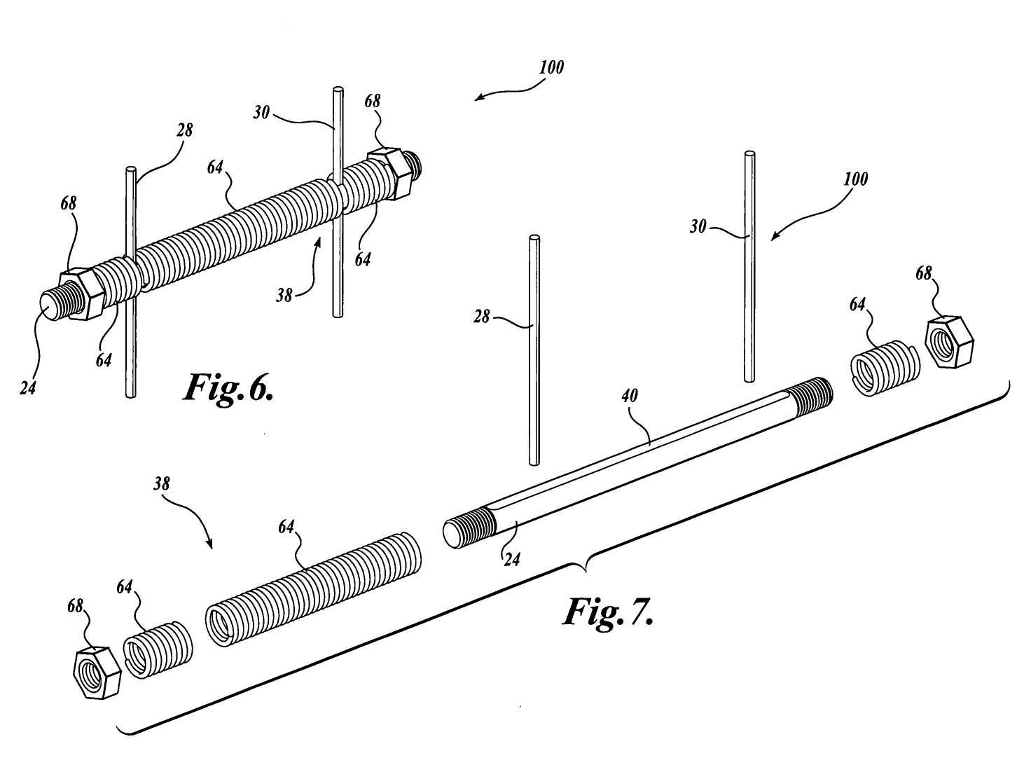

FIGS. 6 and 7 depict another embodiment of an external fixation device constructed in accordance with aspects of the present invention. The fixation device 100 is substantially similar in construction, materials, and operation as fixation device 20 except for the differences that will now be described. The fixation device 100 utilizes spacer components 64 and securement fasteners 68 as the dynamic securement assembly 38 for coupling the support shafts 28 and 30 to the support member 24. In use, the slight flexure of the support shafts 28 and 30 and/or the slight movement afforded by the coupling between spacer components permits micro-motion between the bone segments during use. One use of the fixation device illustrated in FIGS. 6 and 7 is to injuries to the joint-ligamento taxis/distractions.

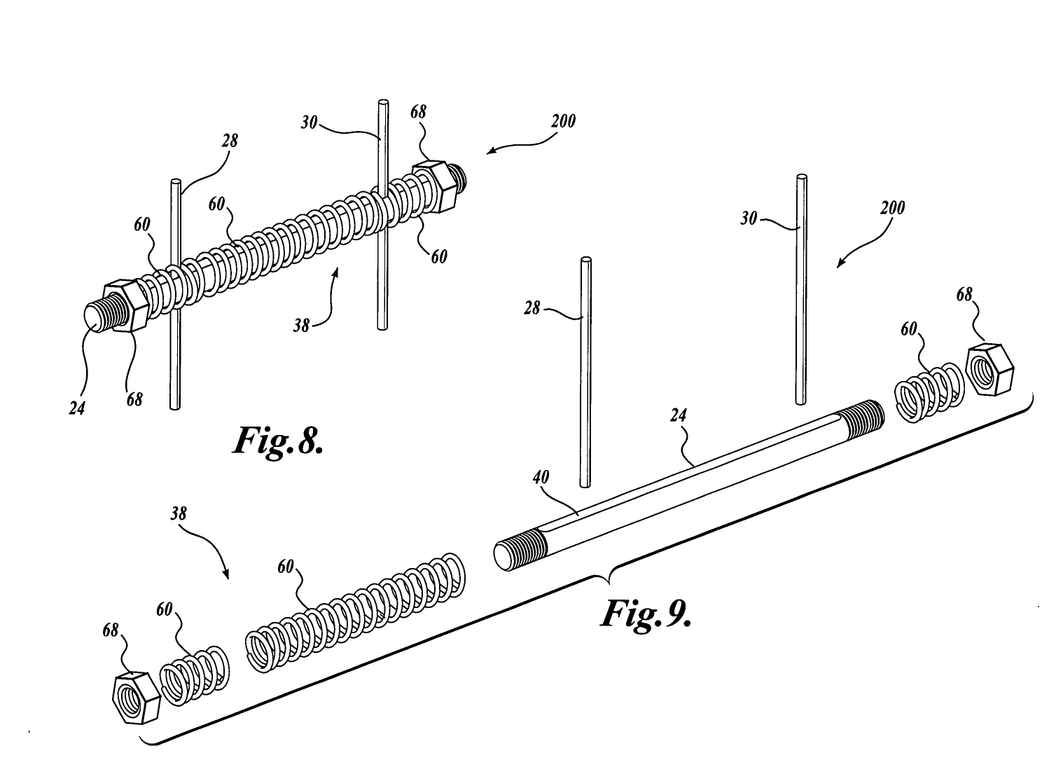

FIGS. 8 and 9 depict another embodiment of an external fixation device constructed in accordance with aspects of the present invention. The fixation device 200 is substantially similar in construction, materials, and operation as fixation device 20 except for the differences that will now be described. The fixation device 200 utilizes biasing components 60 and securement fasteners 68 as the dynamic securement assembly 38 for coupling the support shafts 28 and 30 to the support member 24. In some embodiments, it may be desirable for having the biasing components disposed between the support shafts 28 and 30 (i.e., inner biasing component) exert a force against the shafts 28 and 30 that is greater than the biasing components 60 disposed between the shafts 28 and 30 and the securement fasteners 68, respectively (i.e.; outside biasing components). It will also be appreciated that in some embodiments, the spring constants for the outside biasing components may be either substantially identical or different, depending on application of the fixation device.

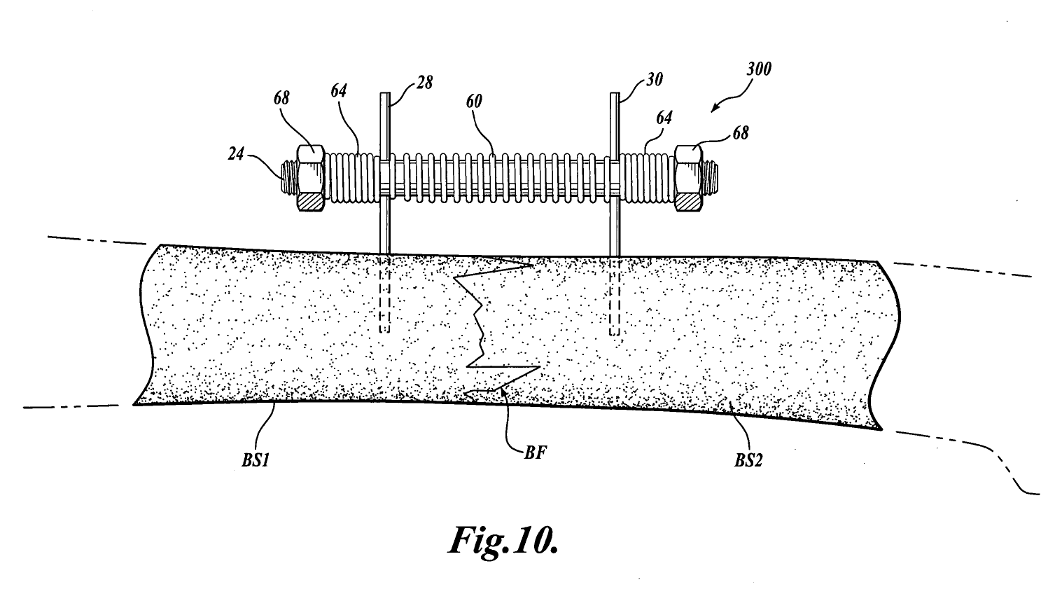

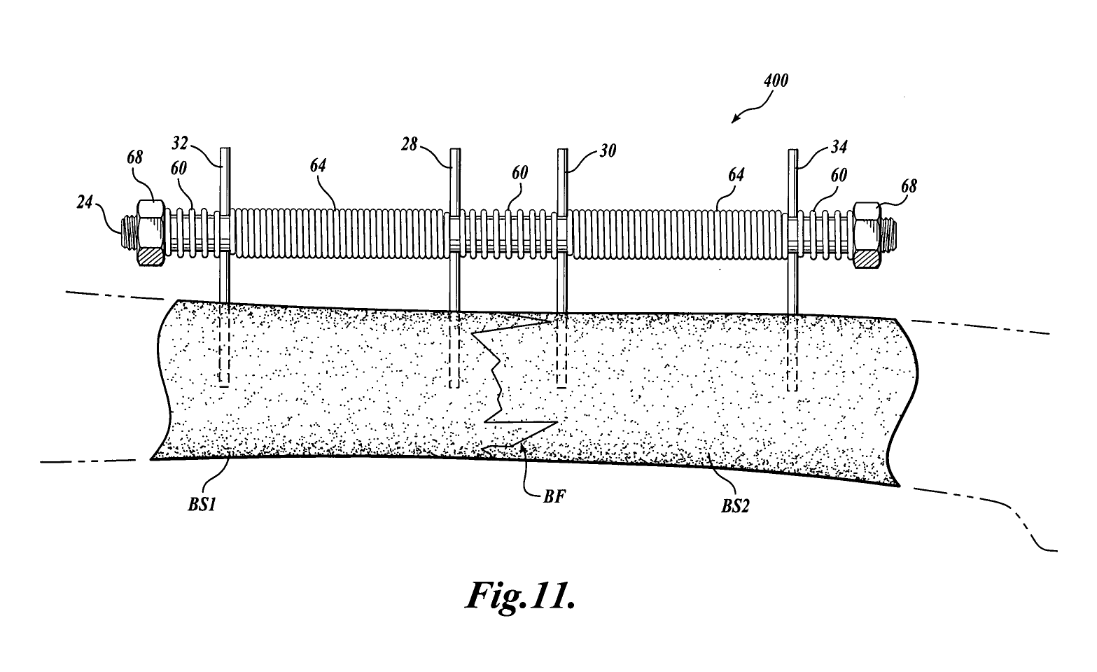

FIG. 10 depicts another embodiment of an external fixation device constructed in accordance with aspects of the present invention. The fixation device 300 is substantially similar in construction, materials, and operation as fixation device 20 except for the differences that will now be described. The fixation device 300 utilizes biasing component 60, spacer components 64, and securement fasteners 68 in the configuration shown as the dynamic securement assembly 38 for coupling the support shafts 28 and 30 to the support member 24.

FIG. 11 is another embodiment of an external fixation device constructed in accordance with aspects of the present invention. The fixation device 400 is substantially similar in construction, materials, and operation as fixation device 20 except for the differences that will now be described. As best shown in FIG. 8, the fixation device 400 utilizes four (4) support shafts 28, 30, 32, and 34 to affix to the first and second bone segments BS1 and BS2. The support shafts 28, 30, 32, and 34 are dynamically secured in a desired position with alternatingly disposed biasing components 60, such as dynamic springs, and spacer components 64, such as static springs. The embodiment illustrated in FIG. 8 is suitable for use in treating displaced or “gap” fractures.

While this embodiment is depicted as using dynamic and static springs in the manner illustrated, it will be appreciated that alternative embodiments are contemplated. For example, in some applications it may be desirous to have any combination of static and/or dynamic springs having identical or different spring constants, as determined by the physician and/or technician.

### EXAMPLE 1

Embodiments of the present invention are currently involved in ongoing human trials for treating bone fractures in small bones of the hand and foot, and have shown a decrease of approximately 25-40% in healing time. The specifications of the components of one embodiment of the fixation device are as follows:

1. Support member—a 4 millimeter (mm) cylindrical rod having a total length of 50 mm and threaded at both ends to 10 mm, and a central slot 1.5 mm wide and 30 mm long;

2. Support shafts—1.4 mm K-wires;

3. Spacer component—a static spring having a 5 mm diameter and a precut length of 50 mm. The thickness of the static spring is 1 mm;

4. Biasing component—two dynamic springs having a 5 mm diameter and a length of 10 mm;

5. Securement fastener—two locking nuts.

The above-identified components were assembled according to the illustrations and description discussed in the embodiments of FIGS. 1-5B.

The embodiments of the fixation device described herein and further contemplated may be used in many different applications, such as fixating small bones of a human's hand or foot. It should be readily apparent that the embodiments of the fixation devices have wide application, and may be used in any situation where substantially stabilizing bone segments in proximity to a fracture and permitting axial and transverse micro-motion between the bone segments is desirable. The fixation devices are equally applicable to all animals including humans, and any bones thereof, a few suitable examples being unstable and displaced phalangeal fractures, displaced metacarpal fractures, and fractures of the bones of the foot. Thus, it should be apparent to those skilled in the art that the embodiments of the fixation devices disclosed herein are meant to be illustrative in nature and not limiting the broadest scope of the invention, as claimed.

While the preferred embodiments of the invention have been illustrated and described, it will be appreciated that various changes can be made therein without departing from the spirit and scope of the invention. For instance, it will be appreciated that the present invention may be applied to both external and internal fixation devices alike. It will also be appreciated that the fixation device of the present invention can be constructed in a manner such that various components, such as the support member, may be reusable or made for a single use, and/or are radio-lucent. It will also be appreciated that the components may be sold unassembled in a kit, and the kit may include various sizes of each or some of the components.

1. A fixation device for treating bone fractures, comprising;
(a) an elongate fixator body having a length;
(b) a first support pin having a proximal end and a distal end, the distal end for coupling to a first bone segment and the proximal end for movably coupling to the elongate fixator body; and
(c) a second support pin having a proximal end and a distal end, the distal end for coupling to the second bone segment and the proximal end for movably coupling to the elongate fixator body;
(d) wherein the proximal end of the first and second support pins are movably secured to the elongate fixator body at selectively adjustable locations so as to define a separation distance therebetween.

2. The fixation device of claim 1 further including a spacer disposed between the first and second support pins to partially determine the separation distance between the first and second support pins.

3. The fixation device of claim 2, wherein the spacer is a biasing component adapted to bias the first support pin away from the second support pin.

4. The fixation device of claim 2 furthering including a fastener for coupling to a distal end of the elongate fixator body.

5. The fixation device of claim 4, wherein the fastener may be selectively positioned along the length of the elongate fixator body to thereby compress the spacer between the first and second support pins.

6. The fixation device of claim 2 further including an outer spacer disposed between the fastener and the first or second support pin.

7. The fixation device of claim 6, wherein the outer spacer is a biasing component adapted to bias the first or second support pin from the fastener.

8. A bone fracture fixation kit, comprising;
(a) an elongate fixator body having a length and a slot disposed substantially along the length;
(b) a first support shaft having a distal end for coupling to a first bone segment of a user and a proximal end for being slidably received within the slot of the elongate fixator body;
(c) a second support shaft having a distal end for coupling to a second bone segment and a proximal end for being coupled to the elongate fixator body; and
(d) at least one of a spacer or a biasing component each having an inner passage for receiving the elongate fixator body at least partially therein, wherein the spacer or biasing component is adapted to be received by the elongate body and be disposed between the first and second support shafts when the first and second support shafts are received by the slot of the elongate fixator body to provide a selected separation therebetween.

9. The kit of claim 8, further comprising a securement fastener for holding one of the support shafts against the spacer or biasing component.

10. The kit of claim 8, further comprising a plurality of spacers, biasing components, or combinations thereof, and at least two securement fasteners.

11. The kit of claim 10, wherein the spacers are static springs and the biasing components are dynamic springs.

12. The kit of claim 10, wherein the securement fasteners are threaded locking nuts.

13. A fixation device, comprising:
a support member;
a plurality of support shafts movably associated with the support member; and
at least one biasing component positioned between the support shafts by the support member, wherein the biasing component and the support member movably interconnect the support shafts at a variable separation distance so as to provide controlled interaction between the support shafts upon application of a sufficient force on one of the shafts.

14. The fixation device of claim 13, wherein the fixation device is configured for use external to the skin surface of a patient.

15. The fixation device of claim 13, wherein the longitudinal axis of each support shaft is substantially perpendicular to the longitudinal axis of the support member.

16. A fixation device, comprising:
a support member, wherein the support member includes at least one slot extending
through the side of the support member;
first and second support shafts extending through the slot of the support member, the slot and the support shafts being configured and dimensioned to allow movement of the support shafts in a longitudinal direction with respect to the support member; and
a coupling assembly positioned along the support member, wherein the coupling assembly is configured and arranged to 1) releasably couple the support shafts to the support member at a selectable separation distance; 2) apply a force against the support shafts; and 3) permit controlled movement of the first support shaft with respect to the second support shaft.

17. The fixation device of claim 16, wherein the force applied against the support shafts is variable.

18. The fixation device of claim 16, wherein the longitudinal axis of each support shaft is substantially perpendicular to the longitudinal axis of the support member.

19. The fixation device of claim 16, wherein the coupling assembly includes springs to apply force against the support shafts.

20. The fixation device of claim 16, wherein the coupling assembly includes at least one selectively positionable coupler, the position of the coupler partially determining the magnitude of the applied force against the support shafts.

21. The fixation device of claim 20, wherein the coupler is a threaded fastener or clamp.

22. A fixation device used to treat bone fractures, comprising:
a support member having a longitudinal axis; and
at least two support shafts having distal ends adapted to be connected to bone segments, the support shafts being slidably coupled to the support member in a spaced-apart manner, the support shafts being oriented substantially transverse to the longitudinal axis of the support member;
wherein the support shafts are biased inwardly along the longitudinal axis of the support member.

23. The device of claim 22, further comprising biasing structure operably associated with the support member and the support shafts, the biasing structure configured to bias the support shafts inwardly along the longitudinal axis of the support member.

24. A fixation device used to treat bone fractures, comprising:
a fixator body having a longitudinal axis;
first and second support pins each having distal ends adapted to be connected to a bone segment; and
means for dynamically coupling the support pins to the fixator body in a selectively adjustable manner and for controlling the movement of the first support pin with respect to the second support pin.

25. A method of treating a bone fracture located between first and second bone segments, comprising:
a) inserting first and second support shafts into the first and second bone segments, the first and second bone segments being disposed on the opposite sides of the bone fracture; and
b) elastically interconnecting the first support shaft to the second support shaft.

26. A method of fixating two bone segments having a bone fracture disposed therebetween, comprising:
a) releasably securing a first support pin to the first bone segment;
b) releasably securing a second support pin to the second bone segment; and
c) dynamically coupling the first and second support pins to a support member in a selectively adjustable manner, the dynamic coupling also providing controlled movement of the first support pin with respect to the second support pin upon application of an external force to one of the bone segments.

27. A method for externally fixating a bone fracture, comprising:
a) inserting a first pin into a first bone segment adjacent the bone fracture;
b) inserting a second pin into a second bone segment adjacent the bone fracture, the second bone segment being disposed on the opposite side of the bone fracture from the first bone segment;
c) applying compressive forces against the first and second support pins; and
d) providing micro-motion in proximity of the bone fracture to promote healing.
